SEQUENCE LISTING (24 sequences)

SEQ ID NO: 1 - DNA, 11190 bp, Homo sapiens
cagaggacaacatagataggttcggagactactcagagaaagggagagagaatgctaggttatctttcagctgttttagctgcttattgttccagctttagccaccatctgactgcaattgtatgagccagaaatagccaggtaaaattgtcttcccaaattcctagtgcacaaaaactgtcagaggctgggggcagtggctcactcctgtaatcccagcagcactttgggaagccagggtgggagaactgcttgagcccaggagttagaggcaacataacaacaactcatctctaatgaaaaaaaaaaaaaaagaaagaaaaaggccaggcacagtggctcacgcctgtaatcccagcactttaggaagccaaggagggtggatcatgaggtcaggagtttgagaccagcctggccaatatggtgaaaccccgtctctactaaaaacacaaaaatcagccacgcatagtggctggtgcctgtaatcacagctactcaggaggctaaggcaggagaatcacttgaatccgggaggcagaggttgcagtgagccgagattgcaccactgcatttcagcctgggcagcagagtgggactgtctcacattaattaattaattgattaaatgctagaagaaaatatggtcaaaatcttttataactcttggagtgaggaaggcaatctcggctcactacaacttccacctcccgggttcaaatgattctcctgcctcagcctcctgagtagctgggattacaggcacccaccaccatgcccggctaatttttgtatttttagtagagacgaggtttcgccatgttggccaagcttgtcttaaactcctgacctcaggtgatccacccacctcagcctcccaaattgctgggattacaggcatgagccaccatgccccgcctaattttaaaaatgtttataaagacagagtcttaccacgttgtccagacctgtctcaaactcctgggctcaagtcattgtcttgtctcagcctcccaaagttctgggactacaggtgtgcaccaccacacctggctaattttgtttattttttatagagtgagggtctccctgtgttgtccagggtgtctcaaactcctgggctcaagtggtcctcctgcctcagcctcccaaagtgctgggattataggcataagccactgcacctggccccagtgtgtgtcttctgaggctaagtcataaagagattgcggcttctattttgcttcctcttagatcactcattctgggggaaaccaacaataaataacaggaacacaccaggagatcagagaaagctgagatacaggcctaatgcccagtcctctgtattcatttctaattgctgctgtaacaagttactgcacatttaatgatgtaaaacaacacaaatttatcttacagttctgtagcttagaagtctgacagagtctcactgggctgaaatcaagatgttgatagttccttcccggtgactctagagtgagaatccagttctttaccctttcttgcctttagaggccacccgtatttattagctcacaatccccttcctccatcttcaaaccagaaacattgcagctctctgtgtcttttttctttactcacatctccctctgactttcttctgccatcctccttcattttttaaggacccctgtgggccaggcatggtggcttatgcctgtaatcccagcactttgagaggcggaggcaggcgggtcacctgaggtcaggagttccagaccagcctggccaacatggcaaaaccccatctccactgaaaatacaaaaattgggccaggcacggcggctcacacccgtaatccccatactttgagaggctgaggcaggtggatcacttgaggtcaggagttcaagaccagcctggccaacatggtgaaatcccgtctctactaaaaaaaaaaaaaattacaataattagtcaggcgtggtggccagcacctataatcccagctacttgggaggttgaggcacaagaattgcttgaacccgagagggggaggttgcagtgagctgagattgcaccaccgtactccagcctggatgacagagcaagactctgtctcaaaataaaaattaagataaataataaaaaataaataaaaatcagcaaattaggccaggtgtggtggtggctcatgcctgtactcccagcactttgaggtgggaaggttgcttgaaaccagaagtttagaccagcctgggaaacaaagtgagaccccatctctacaaaaataaaaataaattagccaggtgtggtgggacgtacttgtactcctagctactagggaggctgaggtgcaagaatcgcttgaacctgggagacggaggttgcagtgagttatgacagtgccactacactccagcctgggtgtcaagggtctttgaaaaaataaacaaaataggctgggcgtggtggcttacacttgtaatcccagcactttgggaggctgaggcaggcagatcacaaagtcaggagttcgagaccagcctggccaacacagtgaaaccccgtctctactaaaaatacaaaaattagccaggcatggttgcacgcgcctgtagtcccaactgctcgggaggctgaggcaggagaatggcttgaacctgggggggggcggaagttataatgagccaagattgtgccactgcactccagcctaggcaacagagcgagactccgtctcaaaaataaaaataaaaataaatcaatagagcctggtatgatggctcacgcctataatcccagcactttgggaggcccaggtgggtggatcatctgaggtcaggagtttgagaccagcctgaccaacatggagaaaccccatctctactaaaaatacaaaaaattagccaggcgtggtggcacatgcctataatcccagctactcaggagcctgaggcaggagaatcgctttaaccggggaggtggaggttgcggtgagtcgagatagcaccattgcattctagcctgggcaacaagagcaaaactccatctcaaaaataaataaataaatagatagataggctgggcacggtggctcacgcctgtaatcccagcactttgggaggccgaggtgggcggatcacctgaggtcaggagttcaagaccagcctggccaatatagcgaaaccctgtctctactaaaaatttaaaaaattagccaggcatgatggcgggtgcctgtaatcccagctactcgggaggctgaggcaggagaatctctcgaacctggcaggcggaggttgcagtgagccgagatcacaccattgcactccagttgggcaacaagagcgaaactccacctcaaaaacaaaataaaataaaataaaatcaggagattggtcagcttaattccatctgcaaccttaattcccttttggccatttaacctatcatattcacaagttccagggattcatgcagggacatctttggtgaccattattctgtctaccacactctctagaagagagtagatgtgagaaacagcatctggagagggaggagccagcaggagagggaaatatcacagacgccagaggaagagagttcccagaagggtaggtgcaacagtaagggatccctctgggaagtcaagcaagagaagacctgaaaaacaaccattggccgggtgcggtggctcacgcctataatcctagcattttgggaggccgaggtgggtggatcacttgaggtcaggagtttaagacaagcctggccaacatggtgaaaccctgtctctactaaaaatacaaaaattagccagacatggtggcaggcacctgtagtccaaactacttgggaggctgaggcacgagaaccacttgaaccctggaggcggaagttgcagtgagccgagatggcaccgctgcactccagcctgggcgacagagtgagactccgtctcaaaataagtaaataaataaataaataaccattgggtctagcgtcttggtgacaccagccactaggtgtcagggcaatgctggaggcagcagctagtttatggcgtgggcagcaaggaagaagtgaggaagtagagactgggagtgtagaatctttccaaaaacttgaatgtggaggggtagatgggataatgccgggagaggaggctggtgaaggacctcttagtgaagggagatttgttgctgtttctgagatgggaaagagaaacaagttgcagtgtagatggggaggatggagaggttgaaggtgtaggagagatgggtcaccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccacggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggactttcgctcttgttgtccaggctggagtgcaatggcacaatttcggctcaccacaacctccacctcccaggttcaagtgattctcctgcctcagcctcccgagtagctgggattacaggcatgcgccaccaggcctggctaattttgtatttttagtagagacagtgtttctctatgttggtcaggctggtctcgcactcccgacctcaggtgatctgcctgctttggcctcccaaagtgctgggattacaggcgtgagccaccatgcccggccagcatctataatatatgctaaaccttacccacagctgcctctgtcttggggtcagggttcttgaatggtctgggccagcctgagttaggagtgggaagaggaagaggaggcacgcgccaaagtgttcttcccgcctgaccgagtcctctgcgcctatgaggaaggtgggcaccgctgtcaccctgtttcccaggaagccagcaggcccagggagatactgggatttgccaaaggctcacagcgagcaagcagcagtgggtgctggctcctcaccactcagcctggcacacactgtctctgagcagacctgggagaaggcatgtgaaacccacctagccctggctggaagagaagtgggggagtggaaatgaaaagtggggactgggaggagctgcccacgtgccctccatggttccaggatggcactttcatgctgggagtgagcttgccctgtgccaggctgtcattgagacgttttccattcaacaactgtttctcagacaaggcacaggcaggttagctctcccaggccactcaagtagtaaggaaacagatttaaacccagacaatttggctccacggctaaggcccagagaggggtggtgacaagcctagggtcacacagccacagccccagagctaggaactgctggctagggcaagcagacagctgaatcctagccaactggaggtggagcagaggtggagccagggagacccaggtggcctc

SEQ ID NO: 2 - DNA, 23 bp, Artificial Sequence
  Description of Artificial Sequence Synthetic primer
cagaagggtaggtgcaacagtaa

SEQ ID NO: 3 - DNA, 23 bp, Artificial Sequence
  Description of Artificial Sequence Synthetic primer
cactgcaacttgtttctctttcc

SEQ ID NO: 4 - DNA, 73 bp, Artificial Sequence
  Description of Artificial Sequence Synthetic oligonucleotide
gtcaagcaagagaagacctgaaaaacaaccattggccaggtgcggtggctcacgcctataatcctagcatttt

SEQ ID NO: 5 - DNA, 70 bp, Artificial Sequence
  Description of Artificial Sequence Synthetic oligonucleotide
atgacgctgcggaattttgggatggggaagaggagcattgaggaccgtgttcaagaggaagcccgctgcc

SEQ ID NO: 6 - DNA, 81 bp, Artificial Sequence
  Description of Artificial Sequence Synthetic oligonucleotide
cacatgccctacacagatgctgtggtgcacgaggtccagagatacattgaccggcgccccaccagcctgccccatgcagtg

SEQ ID NO: 7 - DNA, 11 bp, Artificial Sequence
  Description of Artificial Sequence Synthetic oligonucleotide
tggccgggtgc

SEQ ID NO: 8 - DNA, 32 bp, Artificial Sequence
  Description of Artificial Sequence Synthetic primer
ccgctcgagtagatgtgagaaacagcatctgg

SEQ ID NO: 9 - DNA, 27 bp, Artificial Sequence
  Description of Artificial Sequence Synthetic primer
cccaagcttaaaccagccacggagcag

SEQ ID NO: 10 - DNA, 20 bp, Artificial Sequence
  Description of Artificial Sequence Synthetic primer
gccagcaggagagggaaata

SEQ ID NO: 11 - DNA, 20 bp, Artificial Sequence
  Description of Artificial Sequence Synthetic primer
agtttggactacaggtgcct

SEQ ID NO: 12 - DNA, 360 bp, Homo sapiens
gaaacagcatctggagagggaggagccagcaggagagggaaatatcacagacgccagaggaagagagttcccagaagggtaggtgcaacagtaagggatccctctgggaagtcaagcaagagaagacctgaaaaacaaccattggccgggtgcggtggctcacgcctataatcctagcattttgggaggccgaggtgggtggatcacttgaggtcaggagtttaagacaagcctggccaacatggtgaaaccctgtctctactaaaaatacaaaaattagccagacatggtggcaggcacctgtagtccaaactacttgggaggctgaggcacgagaaccacttgaaccctggaggcgga

SEQ ID NO: 13 - DNA, 30 bp, Artificial Sequence
  Description of Artificial Sequence Synthetic primer
aataataatatgcacgaggtccagagatgc

SEQ ID NO: 14 - DNA, 22 bp, Artificial Sequence
  Description of Artificial Sequence Synthetic primer
gatactatgaatttgggacttc

SEQ ID NO: 15 - DNA, 30 bp, Artificial Sequence
  Description of Artificial Sequence Synthetic primer
aataataatatgcacgaggtccagaggtac

SEQ ID NO: 16 - DNA, 420 bp, Homo sapiens
aagtttgcatatacttccagcactataatttaaatttataatgatgtttggataccttcatgattcatatacccctgaattgctacaacaaatgtgccatttttctccttttccatcagtttttacttgtgtcttatcagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaaggtaagtttgtttctcctacactgcaactccatgttttcgaagtccccaaattcatagtatcatttttaaacctctaccatcaccgggtgaga

SEQ ID NO: 17 - DNA, 23 bp, Artificial Sequence
  Description of Artificial Sequence Synthetic primer
gaattgctacaacaaatgtgcca

SEQ ID NO: 18 - DNA, 24 bp, Artificial Sequence
  Description of Artificial Sequence Synthetic primer
gcagtgtaggagaaacaaacttac

SEQ ID NO: 19 - DNA, 20 bp, Artificial Sequence
  Description of Artificial Sequence Synthetic primer
caagttccagggattcatgc

SEQ ID NO: 20 - DNA, 18 bp, Artificial Sequence
  Description of Artificial Sequence Synthetic primer
gtgccatctcggctcact

SEQ ID NO: 21 - DNA, 23 bp, Artificial Sequence
  Description of Artificial Sequence Synthetic primer
gccagcaggagagggaaatatca

SEQ ID NO: 22 - DNA, 23 bp, Artificial Sequence
  Description of Artificial Sequence Synthetic primer
ctgccaccatgtctggctaattt

SEQ ID NO: 23 - DNA, 23 bp, Artificial Sequence
  Description of Artificial Sequence Synthetic primer
tattctgtctaccacactctcta

SEQ ID NO: 24 - DNA, 23 bp, Artificial Sequence
  Description of Artificial Sequence Synthetic primer
cccaagtagtttggactacaggt

(19) United States
(12) Patent Application Publication
(10) Pub. No.: US 2007/0298426 A1
(43) Pub. Date: Dec. 27, 2007

(54) WARFARIN DOSAGE PREDICTION

(75) Inventors: Yuan-Tsong Chen, Taipei (TW); Chih-Lung Shen, Taipei (TW); Chi-Feng Chang, Taipei (TW); Ming Ta Michael Lee, Taipei (TW); Jen-Chi Hsu, Taipei (TW); Liang-Suei Lu, Taipei (TW); Ming-Shen Wen, Taipei (TW)

Correspondence Address: OCCHIUTI ROHLICEK & TSAO, LLP, 10 FAWCETT STREET, CAMBRIDGE, MA, 02138, US

(73) Assignees: Academia Sinica, Taipei (TW); PharmiGene Inc., Taipei (TW)

(21) Appl. No.: 11/757,860

(22) Filed: Jun. 4, 2007

Related U.S. Application Data
(60) Provisional application No. 60/810,289, filed on Jun. 2, 2006.
(60) Provisional application No. 60/910,978, filed on Apr. 10, 2007.

(51) Int. Cl. C12Q 1/68; A61K 31/366

(52) U.S. Cl. 435/6; 514/457

(57) ABSTRACT

This invention relates to predicting a patient's warfarin dose based on the nucleotide at position −1639 of the VKORC1 gene and the genotype of the CYP2C9 gene in that patient. The warfarin dose so predicted can be further adjusted according to the patient's non-genetic factors, e.g., age, body surface area, medical conditions, and use or non-use of certain drugs.

### CROSS-REFERENCE TO RELATED APPLICATIONS

This application claims the benefit of U.S. patent application No. 60/810,289, filed Jun. 2, 2006, and U.S. patent application No. 60/910,978, filed Apr. 10, 2007. The contents of these two applications are incorporated herein by their entireties.

### BACKGROUND

Warfarin is a widely prescribed anticoagulant for preventing thromboembolism in patients with deep vein thrombosis, atrial fibrillation, or prosthetic heart valve replacement. See Hirsh, American Heart Journal, 123(4 Pt 2):1115-1122, 1992; Laupacis et al., Chest, 108(4 Suppl.):352S-359S, 1995; Stein et al., Chest. 108(4 Suppl.):371S-379S, 1995; and Hirsh et al., Chest, 119(1 Suppl.):8S-21S, 2002. However, warfarin treatment is problematic because its over-dosage can cause serious complications, e.g., bleeding. See Bogousslavsky et al., Acta. Neurol, Scand., 71(6):464-471, 1985; Landefeld et al., Am. J. Med., 95(3):315-328, 1993; Gullov et al., J. Thromb. Thrombolysis, 1(1):17-25, 1994; and Beyth et al., Annals of Internal Medicine, 133(9):687-695, 2000. Much effort has been devoted to monitoring the safety of this drug. Currently, the warfarin dosage for a patient must be adjusted based on serial determinations of blood prothrombin time using standardized international normalized ratio (INR).

It is difficult to prescribe the warfarin dose required by a patient for two reasons: (1) a patient's warfarin dose requirement is highly variable, both inter-individually and inter-ethnically (see Loebsterin et al., Clin. Pharmacol. Ther., 70(2):159-164, 2001; Takahashi et al., Clin. Pharmacol. Ther., 73(3):253-263, 2003; and Zhao et al., Clin. Pharmacol. Ther., 76(3):210-219, 2004; and (2) the dose range for each patient is very narrow.

Some studies suggested that a patient's warfarin dose requirement might be affected by his or her genetic background. For example, Asians generally require a much lower maintenance dose than Caucasians and Hispanics. See Takahashi et al., 2003; Zhao et al., 2004; Yu et al., QJM, 89 :127-135, 1996; and Xie et al., Annu. Rev. Pharmacol., 2001.

Cytochrome P450, subfamily IIC, polypeptide 9 (CYP2C9) is an enzyme that metabolizes warfarin (see Kaminsky et al., Drug Metab. Dispos., 12 :470-477, 1984; Rettie et al., Chemical Research in Toxicology, 5(1) :54-59, 1992; and Kaminsky et al., Mol. Pharmacol., 43 :234-239, 1993). Polymorphisms of this gene were found to be associated with warfarin sensitivity/resistance. More specifically, the polypeptide encoded by CYP2C9 variants (taking CYP2C9*1 as the wild type) CYP2C9*2 and CYP2C9*3 exhibit reduced enzymatic activity, resulting in a lower warfarin dose requirement. See Furuya et al., Pharmacogenetics, 5(6):389-392, 1995. However, as the frequencies of these two CYP2C9 variants in Asians are low, the polymorphisms of this gene cannot fully explain the variations of warfarin dose requirement, in particular, the low dose requirement in Asian populations. See Yuan et al., Human Molecular Genetics, 14(13):1745-1751, 2005 and Nasu et al., Pharmacogenetics, 7(5)405-409, 1997.

Vitamin K epoxide reductase complex, subunit 1 (VKORC1) is an enzyme involved in the blood-clotting pathway. Warfarin inhibits this enzyme by reducing the regeneration of vitamin K and thus exerting its anti-coagulation effect. See Bell et al., Nature, 237(5349):32-33, 1972 and Wallin et al., J. Clin. Invest., 76(5):1879-1884, 1985. It is suggested that polymorphisms of this gene are associated with warfarin sensitivity/resistance. See D'Andrea et al., Blood, 105(2):645-649, 2005; Rider et al., N. Engl. J. Med., 352(22):2285-2293, 2005; Obayashi et al., Clin. Pharmacol. Ther., 80(2):169-178, 2006, and Bodin et al., Blood, 106(1):135-140, 2005.

### SUMMARY OF THE INVENTION

This invention is based on the unexpected finding that the single nucleotide polymorphism (SNP) at position −1639 in the VKORC1 gene and the genotype of the CYP2C9 gene are associated with warfarin sensitivity/resistance.

In one aspect, this invention provides a method of predicting a warfarin dose for a patient based on his or her genotype. This method includes the following steps: (1) determining the nucleotide at the −1639 position of the VKORC1 gene of the patient, (2) examining the sequence of CYP2C9 gene of the patient, and (3) predicting a warfarin dose for the patient based on the nucleotide at the −1639 position of the VKORC1 gene and the CYP2C9 gene sequence. The warfarin dosage is the in the range of 4.5-6.5 mg (e.g., 5 mg) per day for a patient carrying homozygous GG at the −1639 position of the VKORC1 gene and carrying CYP2C9*1/*1. If the patient has homozygous GG at the −1639 position and has CYP2C9*1/*3 or CYP2C9*1/*2, the warfarin dosage ranges from 3.25-4.25 mg (e.g., 3.75 mg) per day. If the patient has homozygous GG at the −1639 position and has CYP2C9*2/*2, CYP2C9*2/*3, or CYP2C9*3/*3, the warfarin dosage ranges from 3.5-4.0 mg (e.g., 3.75 mg) per day. If the patient has heterozygous AG at the −1639 position and has CYP2C9*1/*1, the warfarin dosage ranges from 3.25-4.0 mg (e.g., 3.75 mg) per day. If the patient has heterozygous AG at the −1639 position and has CYP2C9*1/*3 or CYP2C9*1/*2, the warfarin dosage is in the range of 2.5-3.0 mg (e.g., 2.5 mg) per day. If the patient has heterozygous AG at the −1639 position and has CYP2C9*2/*2, CYP2C9*2/3, or CYP2C9*3/*3, or has homozygous AA at position −1639 and has CYP2C9*1/*1, the warfarin dosage is in the range of 2.0-3.0 mg (e.g., 2.5 mg) per day. If the patient has homozygous AA at position −1639 of the VKORC1 gene and has CYP2C9*1/*3 or CYP2C9*1/*2, the warfarin dosage ranges from 1.0-1.5 mg (e.g., 1.25 mg) per day. If the patient has homozygous AA at position −1639 of the VKORC1 gene and has CYP2C9*2/*2, CYP2C9*2/*3, or CYP2C9*3/*3, the warfarin dosage is in the range of 0.75-1.5 mg (e.g., 1.25 mg) per day.

The warfarin dosage predicted based on a patient's genotype (genetic-based dosage) can be further adjusted according to various non-genetic factors of the patient, e.g., age, body surface area, or weight, medical conditions (e.g., hypertension or diabetes), use or non-use of a drug that interferes with CYP2C9 activity or affects VKORC1 expression, or a combination thereof. The drug can be for treating a cardiovascular disease or hypercholesterolemia (e.g., aminodarone or rosuvastatin). The warfarin dosage can be adjusted as follows: A+(B×genetic-based dosage)+(C×age)+(D×body surface area). In this alogrism, A is in the range of −2 −0 (e.g., −1 to −0.5). B in the range of 0.5-1.0 (.e., 0.7 to 0.8), C in the range of −0.1-0.015 (e.g., −0.05 to 0.01), D in the range of 0-5 (e.g., 0.5 to 1.5). The number A can be adjusted based on the patient's medical conditions and use or non-use of certain drugs. In one example, the dosage of warfarin is adjusted as follows: −0.432+(0.769×genetic-based dosage)×(0.015×age)+(1.125×body surface area).

In another aspect, this invention provides a method for determining a final warfarin dosage for a patient. This method includes (1) predicting a warfarin dosage based on the nucleotide at the −1639 position of the VKORC1 gene and the genotype of the CYP2C9 gene, (2) administering to the patient the warfarin at the predicted dosage, (3) monitoring the patient's therapeutic INR after the administration, and (4) adjusting the warfarin dosage until the INR value falls in the range of 1.7-3. If the dosage at which the patient's INR falls in the range of 1.7-3 for at least two consecutive INR measurements, this dosage is determined to be the final warfarin dosage for the patient.

In yet another aspect, this invention features a kit for predicting a warfarin dosage of a patient based on his or her genotype. This kit can contain a first probe for detecting the nucleotide at position −1639 of the VKORC1 gene and a second probe for detecting position c.1075 of the CYP2C9 gene. Optionally, the kit can further contain a third probe for detecting the position c.430 of the CYP2C9 gene. Each of these probes can be an oligonucleotide or a pair of PCR primers.

The term “warfarin” encompasses coumarin derivatives having an anticoagulant activity. A preferred embodiment, warfarin, is 4-hydroxy-3-(3-oxo-1-phenylbutyl)-2H-1-benzopyran-2one (i.e., 3-α-phenyl-β-acetylethyl-4-hydroxycoumarin). The current commercial product is a racemic mixture of the R-isomer and the S-isomer. The term “warfarin” refers to the R-isomer, or the S-isomer, or any racemic mixture, or any salt thereof. Specifically includes s warfarin are Coumadin, Marevan, Panwarfin, Prothromadin, Tintorane, Warfilone, Waran, Athrombin-K, warfarin-deanol, Adoisine, warfarin acid, Coumafene, Zoocoumarin, Phenprocoumon, Dicumarol, Brodifacoum, Diphenadione, Chlorophacinone, Bromadiolone, and Acenocoumarol.

An “oligonucleotide,” as used herein, is a nucleic acid containing 2 to 200 (e.g., 10, 20, 30, 40, 50, 75, 100, or 150) nucleotides. An oligonucleotide can be used as a hydrolysis probe or a hybridization probe. It also can be used as a PCR primer.

A hybridization “probe” is an oligonucleotide that binds in a base-specific manner to a nucleic acid of interest. Such probes can include peptide nucleic acids. A probe is of a suitable length such that it specifically hybridizes to the target nucleic acid. The length of a probe varies depending upon the hybridization method in which it is used. Such optimizations are known to a skilled artisan. Suitable probes typically have lengths ranging from 8 nucleotides to 100 nucleotides in length, e.g., 8-20, 10-30, 15-40, 50-80.

The details of one or more implementations of the invention are set forth in the description below. Other features, objects and advantages of the invention will be apparent from the description and drawings, and from the claims.

### DESCRIPTION OF DRAWINGS

FIG. 1 is a scatter plot of warfarin doses versus the VKORC1 −1639 SNP. Warfarin doses of selected patients are plotted against different SNPs at the VKORC1 promoter −1639 position. *Individulas with CYP2C9 variants including CYP2C9*3, T299A (i.e., amino acid residue 299 changed from T to A), and P382L (i.e., amino acid residue 382 changed from P to L).

FIG. 2 is a plot showing relative measurements of luciferase activity levels in HepG2 cells. pGL3 luciferase reporter contains either the A nucleotide (pGL3-A) or the G nucleotide (pGL3-G) at position −1639 position of the VKORC1 gene. The luciferase activity levels shown in this figure are means of data generated from nine independent experiments. The error bars represent standard deviation. pGL3-basic vector is used as a negative control.

FIG. 3A-3D shows the genomic sequence of the VKORC1 gene (Genbank Accession No. AY587020). The transcription start site is at nucleotide number 5086 (bolded and boxed) in this figure, which is designated as +1 of the gene in the traditional nomenclature. The A of the ATG translation initiation codon (bolded) is at nucleotide number 5312 in this figure, which is recommended by the Human Genome Variation Society to be designated as +1 in the new nomenclature system. The promoter polymorphism described herein is underlined and bolded (nucleotide number 3673 in this sequence), which is at −1413 in the traditional system and −1639 in the new, recommended system.

FIG. 4 is a plot showing the anticoagulation effects in patients treated with warfarin. A: Time to therapeutic INR stratified by dose groups. B: Average weekly PIVKA-II measurements.

FIG. 5 is a plot showing correlation between the predicted doses and the maintenance doses at 12 weeks. The shaded area illustrates where the predicted doses match the maintenance doses.

FIG. 6 shows the nucleotide sequences nearby position −1639 of the VKORC1 gene and positions c.430 and c.1075 of the CYP2C9 gene.

FIG. 7 is a schematic diagram illustrating Competitive Sequence Specific Oligonucleotide-ELISA assay (CSSO-ELISA).

### DETAILED DESCRIPTION OF THE INVENTION

It has been discovered that single nucleotide polymorphisms (SNPs) of the VKORC1 gene (e.g., at the positions −1639) or the genotype of the CYP2C9 is associated with warfarin sensitivity/resistance of a patient. Patients carrying homozygous AA, heterozygous AG, and homozygous GG at the −1639 position of the VKORC1 gene requires the lowest, intermediate, and the highest warfarin dosage, respectively. Thus, the SNP at position −1639 can be used to predict a patient's warfarin dose requirement. It is more accurate to predict the warfarin dosage based on this SNP and the genotype of the CYP2C9 gene. The warfarin dosage thus predicted can be further adjusted based on the patient's non-genetic factors, such as age, body surface area, medical conditions, or concurrent us of another drug (e.g., aminodarone and rosuvastatin).

This present invention provides a method of predicting a warfarin dosage for a patient based on the nucleotide at position −1639 of the VKORC1 gene and the genotype of the CYP2C9 gene.

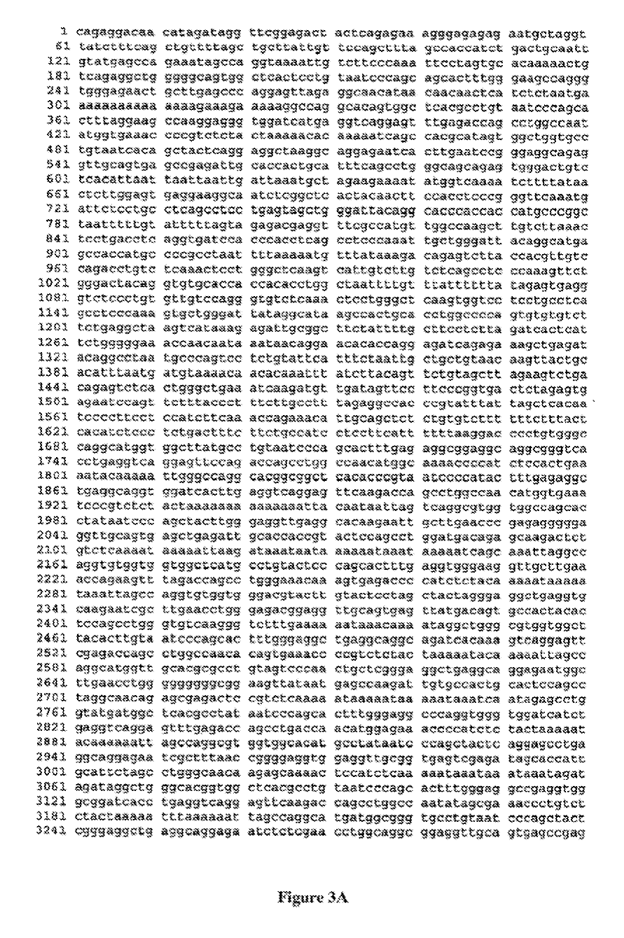

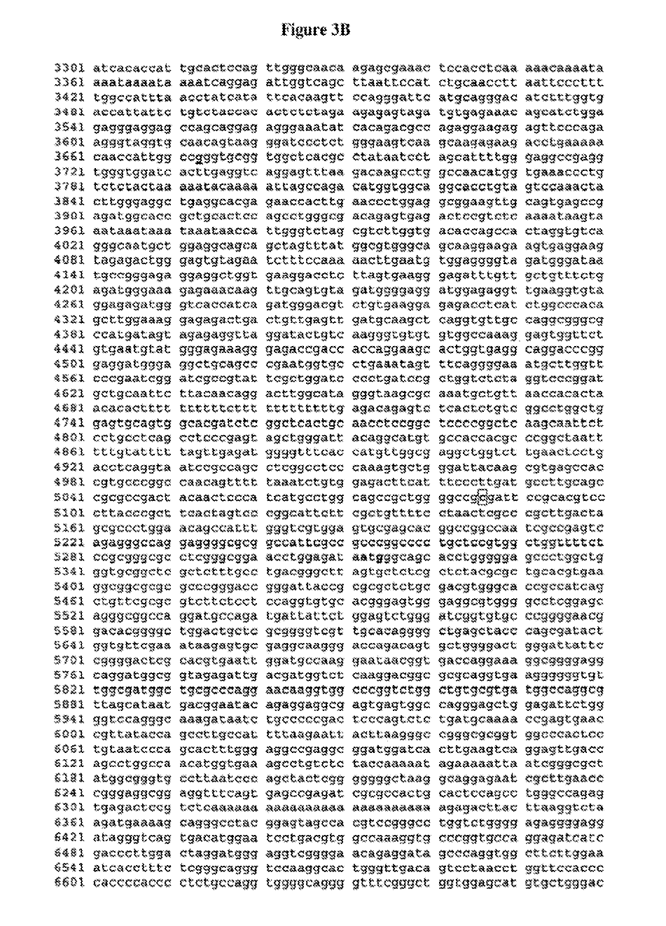

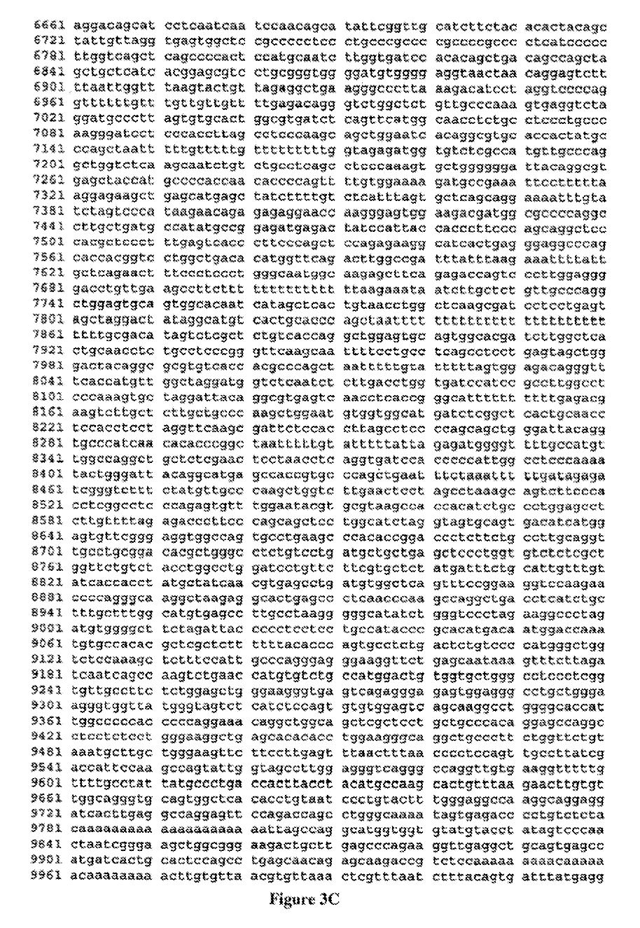

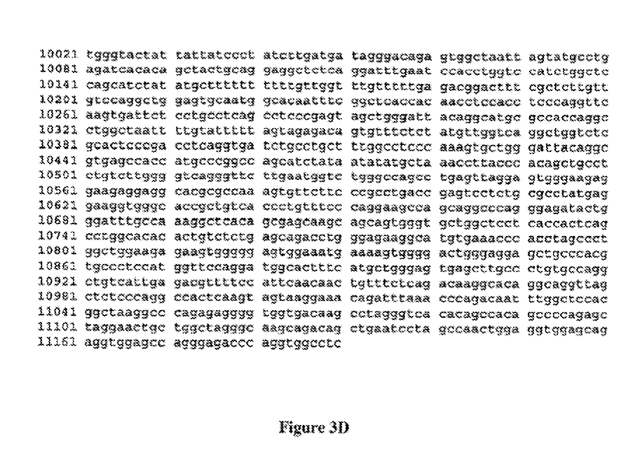

The genomic sequence of the VKORC1 gene is available as Genbank Accession No. AY587020 (SEQ ID NO:1; FIG. 3A-3D). The sequence of VKORC1 isoform 1 mRNA is available as GenBank Accession Number NM_{—}024006.4. In both of these sequences, the start site of transcription is designated as +1. Thus the SNP described herein (indicative of warfarin sensitivity) is located at the −1413 position. However, a recommended by the Human Genome Variation Society (http://www.genomic.unimelb.edu.au/mdi/ mutnomen/), a promoter SNP is described in relation to the A residue of the ATG translation initiation codon. Accordingly, the A of the ATG translation initiation codon of NM_{—}024006.4 or AY587020 is at position +1 and the promoter SNP described herein is at position −1639, i.e., the G>A polymorphism is referred to as “NM_{—}024006.4:c.−1639G>A”. It should be clarified that the −1639 G>A polymorphism (or more precisely “NM_{—}024006.4c.−1639G>A”) described herein is the same as the −1413 G>A polymorphism, which is numbered following the traditional system described above.

The SNPs in the VKORC1 gene and the genotype of the CYP2C9 gene can be determined by methods well known in the art. In general, genomic DNAs can be prepared from a patient's biosample (e.g., blood, saliva, urine, and hair) using DNA purification methods, e.g., PUREGENE DNA purification system from Gentra Systems, Minnesota. Detection of an SNP (e.g., at position −1639) in the VKORC1 gene includes examining the corresponding nucleotide located at either the sense of the anti-sense strand of the DNA. Variants of the CYP2C9 gene can be determined by examining the nucleotides at position c.430 or c.1075. The presence of C at position c.430 or A at position c.1075 indicates that the patient carries wild-type CYP2C9*1. In CYP2C9*2, the nucleotide at position c.430 is T; and in CYP2C9*3, the nucleotide at position c.1075 is C.

Any methods known in the art for genotyping can be used to examine the SNPs in the VKORC1 gene and the CYP2C9 gene, e.g., sequence specific oligonucleotides-hybridization, real-time PCR, CSSP-ELISA (competitive sequence specific probes/oligonucleotides coupled with enzyme-linked immunosorbent assay), RFLP (restriction fragment length polymorphism), and DHPLC (Denaturing High-Performance Liquid Chromatography). When real-time PCR is used for genotyping, PCR products thus obtained can be detected using DNA-binding agents, such as SYBR® Green, or sequence specific probes, which include hydrolysis probes (e.g., TaqMan, Beacons, and Scorpions) and hybridization probes.

In one example, one or more oligonucleotides, e.g., hybridization probes or PCR primers, are used to examine the SNPs described herein. The oligonucleotide can have the sequence TGGCCGGGTGC (SEQ ID NO: ______) (3668 to 3678 of SEQ ID NO:1), or the complement thereof. For the purpose of hybridization, the nucleotide that corresponds to the −1639 position is preferably located near the center of the oligonucleotide.

Hybridization is usually performed under stringent conditions, for instance, at a salt concentration of <1 M and a temperature of at least 25° C. For example, conditions of 5×SSPE and a temperature of 25-30° C., or equivalent conditions thereof, are suitable for single nucleotide-specific probe hybridizations. A low stringent wash after hybridization can be conducted. The wash step is carried out under suitable conditions, e.g., 42° C., 5×SSC, and 0.1% SDS; or 50° C., 2×SSC, and 0.1% SDS. An example of a high-stringent wash condition is 65° C., 0.1×SSC, and 0.1% SDS. Equivalent conditions can be determined by varying one or more of the parameters, as known in the art, while maintaining a similar degree of identity or similarity between the target nucleotide sequence and the primer or probe used.

In addition to the specific polymorphism (e.g., AA, AG or GG at the −1639 position), genetic markers that are linked to each of the specific SNPs can be used to predict the corresponding warfarin sensitivity as well. Equivalent genetic markers near the SNP of interest tend to co-segregate with the SNP of interest. Thus, their presence is indicative of the presence of the SNP of interest, which, in turn, is indicative of the level of warfarin sensitivity.

The VKORC1 −1639 A>G promoter polymorphism is in linkage disequilibrium with the VKORC1 1173 C>T intronic polymorphism recently reported in D'Andrea et al., 2005. This linkage explains the results that Italian patients with the 1173 TT genotype require a lower average daily dose than the CT or CC genotype. 3730 (i.e., rs7294) G>A polymorphism, which is located in the 3′ untranslated region, is also found to be in linkage disequilibrium with −1639 A>G and 1173 C>T in the Chinese population. Specifically, the 3730G allele is associated with the −1639A allele and the 1173T allele. Other equivalent genetic markers of the −1639 SNP include rs9934438 (intron 1), rs8050894 (intron 2) and rs2358612 (intron 2). Thus, −1639A is linked with T at rs9934438, C at rs8050894, T at rs235612 and G at rs7294, while −1639G is linked with C at rs9934438, G at rs8050894, C at rs2359612 and A at rs7294.

The equivalent genetic marker can be any marker, including microsatellites and SNP markers. Useful genetic markers can be 200 kb or shorter from the VKORC1 −1639 position, e.g., 100 kb, 80 kb, 60 kb, 40 kb, 20 kb, 15 kb, 10 kb, 5 kb or shorter from the VKORC1 −1639 position.

A patient's starting warfarin dosage can be predicted based on the nucleotide at position −1639 of the VKORC1 gene and the genotype of CYP2C9. Table 1 below shows the recommended warfarin dosage in connection with a combination of a patient's SNP at position −1639 of the VKORC1 gene and the genotype of the CYP2C9 gene.

TABLE 1
Recommended warfain dose for patients having different combinations
of the genotype of CYP2C9 gene and the VKORC1 −1639 SNP

| | | Recommended Dose |
|---|---|---|
| VKORC1 −1639 G > A | CYP2C9 Genotype | Range (mg/Day) |
| GG | *1/*1 | 4.5˜6.5 |
| GG | (*1/*3) or (*1/*2) | 3.25˜4.25 |
| GG | (*2 or *3)/(*2 or *3) | 3.5˜4.0 |
| AG | *1/*1 | 3.25˜4.0 |
| AG | *1/*3 or (*1/*2) | 2.5˜3.0 |
| AG | (*2 or *3)/(*2 or *3) | 2.0˜3.0 |
| AA | *1/*1 | 2.0˜3.0 |
| AA | *1/*3 or (*1/*2) | 1.0˜1.5 |
| AA | (*2 or *3)/(*2 or *3) | 0.75˜1.5 |

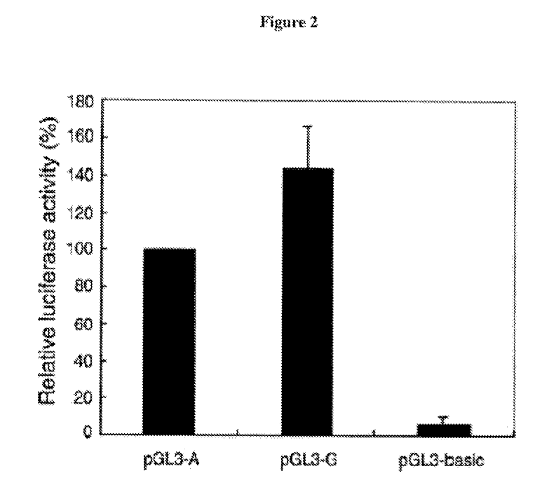

Without being bound by theory, the correlation between the SNP at position −1639 of the VKORC1 gene and warfarin sensitivity can be explained as follows. The −1639 promoter SNP is located in an E-Box (having a consensus sequence of CANNTG.), which is close to (within 200 bp) three additional E-boxes. E-boxes are found to be important elements for mediating cell/tissue specific transcription, e.g., gene expression in muscle, neurons, liver and pancreas. See Massari et al., Mol Cell Biol., 20:429-440 (2000); and Terai et al., Hepatology, 32:357-366, 2000. It is suggested that changing the second nucleotide from A to G as observed at the −1639 site would destroy the E-box consensus sequence and thus alter the promoter activity. This hypothesis is strongly supported by the promoter activity assay results shown in Example 4 below. More specifically, VKORC1 promoter carrying GG at position −1639 shows a promoter activity 44% higher than that carrying AA at position −1639. See FIG. 2.

VKORC1 protein is responsible for regenerating the reduced form of vitamin K, which is required by gamma-carboxylase. Gamma-carboxylation of vitamin K-dependent clotting factors (factor II, VII, IX, and X) is essential for blood clotting. When VKORC1 promoter activity increases, an elevated level of VKORC1 mRNA can lead to higher VKOR activity and thus enhance the efficiency of regeneration of reduced vitamin K. See Rost, et al., Nature, 427:537-541 (2004). Thus, gamma-carboxylation of the vitamin K dependent clotting factors is enhanced due to the higher level of reduced vitamin K. Warfarin acts by blocking clotting factor synthesis, and having more active clotting factors would require more warfarin for its anti-coagulation effect. Liver is the primary organ for the syntheses of vitamin K dependent clotting factors and has the highest expression level of VKORC1. Thus, a 44% change at the level of VKORC1 in the liver is most likely to have a significant impact on the blood clotting process, and in turn, on warfarin dose requirement.

Any nucleotide other than A at the −1639 position of the VKORC1 promoter may destroy the E-box consequence sequence and thus increase promoter activity. An increase in promoter activity, in turn, increases warfarin dose requirement. Thus, the promoter sequence, particularly the nucleotide at the −1639 position, is indicative of warfarin dosage requirement of a patient of any ethnic background, e.g., Mongoloid, Caucasian, and Negroid.

As such, detecting the promoter activity of the VKORC1 gene, levels of the VKORC1 mRNA, or protein can also reflect warfarin dose requirement. A VKORC1 promoter activity, mRNA level, protein level, or VKOR activity that is at least 10%, 15%, 20%, 25%, 30%, or 40% higher than that of a subject having the AA genotype is indicative of a higher warfarin dose requirement.

Methods of determining promoter activities or levels of mRNA/proteins are well known in the art. For example, PCR can be employed to detect mRNA levels and VKORC1-specific antibodies can be used to measure protein levels. Promoter activities can be examined, for example, by isolating the promoter sequence from the subject of interest, linking the promoter sequence to a reporter gene, expressing the reporter gene, and determining the amount of the reporter produced. Methods of measuring VKOR activities are also known in the art.

This invention also features a method of adjusting the predicted warfarin dosage as described above according to that patient's non-genetic factors, e.g., age, body surface area, medical conditions (e.g., hypertension or diabetes), use or none-use of a drug that affects warfarin effect/metabolism, CYP2C9 activity, or VKORC1 expression. Such drugs include those for treating cardiovascular diseases or hypercholesterolemia, e.g., aminodarone or rosuvastatin. A physician would well know how to adjust a patient's warfarin dosage based on these factors. For example, and old (>60) patient or a patient having hypertension/diabetes usually requires relatively lower warfarin dosage. As another example, the warfarin dosage would also need to be lowered if the patient takes aminodarone.

When considering both genetic and non-genetic factors, a patient's warfarin dosage can be predicted as follows: Dose=A+(B×genetic-based dosage)+(C×age)+(D×body surface area), wherein A is in the range of −2 to 0, B in the range of 0.5 to 1.0, C in the range of −0.1 to 0.015, D in the range of 0 to 5. The number A can be adjusted based on the patient's medical conditions and use or non-use of certain drugs.

This invention also features a method of determining a maintenance warfarin dosage for a patient. First, an initial dosage can be predicted as described above, either based solely on genetic factors, or based on the combination of genetic and non-genetic factors. Then, the patient can be administered with warfarin at the predicted dosage. After administration, the patient can be followed-up for his or her INR value, vitamin K status, or over-dose symptoms such as blooding. Warfarin dosage can be adjusted if the patient's INR value does not fall in the range of 1.7-3. If the value is lower than 1.7, the dosage for that patient should be increased. On the other hand, if the value is higher than 3, the dosage should be decreased. Normally, the warfarin dosage can be adjusted by ±1.5 mg each time. When INR>4 is first detected in a patient, in addition to lower warfarin dosage, factors that could cause the adverse events should also be determined. Adverse events includes INR>4 and clinical bleeding, which is defined as major bleeding that requires hospitalization, and occurrence of venous thrombosis/pulmonary embolism. Warfarin administration should be terminate if bleeding events and venous thrombosis/pulmonary embolism occur in a patient.

When a patient's INR values fall in the range of 1.7-3 for two consecutive measurements (e.g., once per week), the dosage on which the patient is administered is his or her maintenance warfarin dosage.

Also within the scope of this invention is a kit containing probes for detecting a patient's SNP at position −1639 of the VKORC1 gene and genotype of the CYP2C9 gene. The term “probe” used herein refers to any substance useful for detecting another substance. Thus, a probe can be an oligonucleotide or conjugated oligonucleotide that specifically hybridizes to a particular region including the nucleotide being examined. The conjugated oligonucleotide refers to an oligonucleotide covalently bound to chromophore or a molecules containing a ligand (e.g., an antigen), which is highly specific to a receptor molecular (e.g., an antibody specific to the antigen). The probe can also be a PCR primer, together with another primer, or a pair of primers, for amplifying a particular region, in which the nucleotide of interest is located. Optionally, the kit can contain a probe that targets an internal control allele, which can be any allele presented in the general population, e.g., GAPDH, β-acting, KIR. Detection of an internal control allele is designed to assure the performance of the kit.

The kit can further include tools and/or reagents for collecting biological samples from patients, as well as those for preparing genomic DNA, cDNAs, or RNAs from the samples. For example, PCR primers for amplifying the relevant regions of the genomic DNA may be included. The kit can also contain probes for genetic factors useful in pharmacogenomic profiling, e.g., thiopurine methyltransferase.

In one example, the kit contains first probe for detecting the nucleotide at position −1639 of the VKORC1 gene and a second probe for detecting the nucleotide at position c.1075 of the CYP2C9 gene. Each of the two probes can be a pair of PCR primers, or a labeled oligonucleotide useful in hybridization assays. The kit can further include a third probe for detecting the nucleotide at position c.430 of the CYP2C9 gene. Optionally, it can include a probe for detecting an internal control allele.

The present invention also features a method of determining whether a given mutation of the VKORC1 gene impacts warfarin dosing. In this method, the promoter activity, mRNA level, protein level, or VKOR activity resulting from the mutation is determined and compared to that of the corresponding wild-type VKORC1 gene. If the promoter activity, mRNA level, protein level or VKOR activity increases or decreases by at least 10%, 15%, 20%, 25%, 30%, 35% or 40% compared to that of the wild-type gene, the physician should consider raising or lowering warfarin dosing.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### EXAMPLE 1

### Warfarin Dosage of Patients with Different Polymorphisms in VKORC1

Sixteen Han Chinese patients were recruited for this study. These patients received warfarin either at low or at high dose from cardiovascular clinics of four major medical centers in Taiwan (National Taiwan University Hospital, Kaohsiung Medical University Hospital, Taipei General Veteran Hospital, and Shin-Kong Wu Ho-Su Memorial Hospital). The mean maintenance dose of warfarin in Chinese patients is 3.3 mg/day, see Yu et al., 1996; and Xie et al., 2001. Among them, 11 patients, who received maintenance dose≦1.5 mg per day, were deemed as warfarin sensitive; and five, who were on warfarin maintenance dose≧6 mg per day, were deemed as warfarin resistant. See Table 2 below.

Genomic DNAs were isolated from these patients using the PUREGENE™ DNA purification system (Gentra systems, Minnesota, USA). CYP2C9 and VKORC1 DNA sequence variants were first determined by direct sequencing (Applied Biosystems 3730 DNA analyzer, Applied Biosystems, Foster City, Calif., USA). Primers were specifically designed for the intron-exon junctions, exons and 2 kbps upstream of the transcription start site for both CYP2C9 and VKORC1 using the Primer3PCR primer program (http://fokker.wi.mit.edu/cgi-bin/primer3/primer3_www.cgi). The primers used to detect variants in the VKORC1 promoter were: 5′-CAGAAGGGTAGGTGCAACAGTAA (SEQ ID NO:2; sense strand located 1.5 kb upstream of the transcription start site) and 5′-CACTGCAACTTGTTTCTCTTTCC (SEQ ID NO:3; anti-sense strand located 0.9 kb upstream of the transcription start site). The polymerase chain reactions (PCR) were performed in a final volume of 25 μl, containing 0.4 μM of each primer, 10 mM Tris-HCl (pH8.3), 50 mM KCl, 1.5 mM MgCl₂, 0.2 mM dNTPs and 1 unit HotStart Taq™ (Qiagen Inc. Valencia, Calif., USA). The amplification reaction was carried out as follows: initial denaturation at 96° C. for 12 min., 34 PCR cycles under the condition of 30 sec at 96° C., 30 sec at 60° C., 40 sec at 72° C. Results thus obtained are summarized below and shown in Table 2.

Sequencing of the coding regions, exon-intron junctions, and the promoter region of the CYP2C9 gene revealed three sequence variants in 4 of the 11 warfarin sensitive patients. The variants were: 1075 A≦C (1359L known as CYP2C9*3), 895 A>G (T299A), and 1145 C>T (P382L). CYP2C9*3 was detected in 3 patients (subjects 1, 3, and 5, see Table 2). Subject 5 carries both CYP2C9*3 and the 895 A>G (T299A) change as previously described, see Zhao et al., 2004. A novel exonic mutation, 1145 C>T (P382L), was detected in the fourth patient (subject 6).

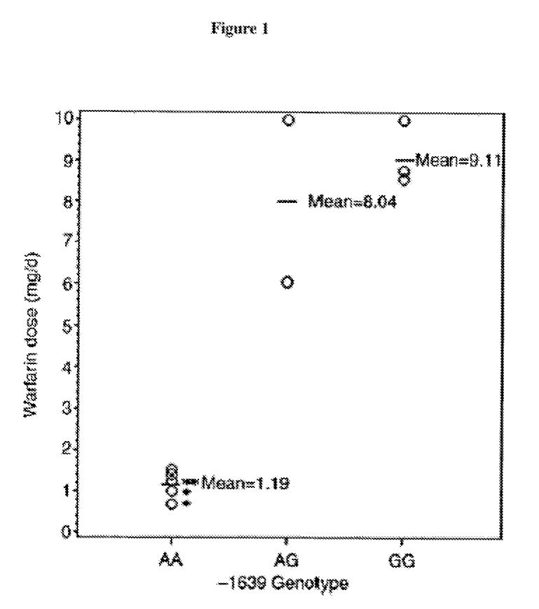

The promoter, coding regions, and the exon-intron junctions of the VKORC1 gene were also sequenced. One variant (3730 G>A), which was located in the 3′ un-translated region (UTR) of VKORC1 was detected. In addition, a single nucleotide polymorphism located within the promoter region, −1639 G>A (or −1413 when numbering transcription start site as +1) was detected. Results showed that this polymorphism is associated with warfarin sensitivity in that all warfarin sensitive patients carry homozygous AA at position −1639. The warfarin-resistant patients, on the other hand, carry either heterozygous AG or homozygous GG (see Table 2). Warfarin doses were plotted against the polymorphisms at position −1639 of the VKORC1 gene. The results demonstrate that patients carrying homozygous AA at this position require the lowest warfarin dose (mean 1.19 mg/day, range 0.71-1.50 mg/day); carrying heterozygous AG require intermediate dose (mean 8.04 mg/day, range 6.07-10 mg/day); and carrying homozygous GG require the highest warfarin dose (means 9.11 mg/day, range 8.57-10 mg/day) (FIG. 1).

In addition to the −1639 G>A position, a polymorphism 1173 C>T located in intron 1, was identified in some of the patients. This polymorphism has been disclosed in D'Andrea et al., Blood, 105:645-649, 2005. The two polymorphisms at position −1639 and 1173 of the VKORC1 gene appeared to be in strong linkage disequilibrium (LD) (see Table 2). In ten Warfarin-sensitive patients, the −1639 AA genotype was found to be associated with homozygous TT at position 1173. In warfarin-resistant patients, heterozygous AG at position −1639 was associated with heterozygous CT at posiion 1173, and homozygous −1639 GG associated with homozygous TT at position 1173 (see Table 2).

### EXAMPLE 2

### Polymorphism at Position VKORC1 −1639 G>A of the VKORC1 Gene in Random Chinese Patients Receiving Warfarin

Randomly selected 104 Chinese patients, 95 normal Chinese controls (selected from a biobank, in which 3312 Han Chinese descendants were recruited based on the geographic distribution across Taiwan) and 92 normal Caucasian controls (Cat. No, HD100CAU, National Institute of General Medical Sciences Human Genetic Cell Repository, Camden, N.J., USA) were participated in this study. The Chinese controls and all participating patients receiving warfarin therapy were unrelated Han Chinese residing in Taiwan. The Han Chinese forms the largest ethnic group in Taiwan, making up roughly 98 percent of the population. None of the participants were aboriginal Taiwanese, which account for the remaining 2% of the Taiwan's population.

The average daily dose of warfarin was calculated from a one-week-period, and the latest international normalized ratio (INR) of each patient was recorded. The randomly recruited 104 patients who received warfarin, regardless of their dose, had a target INR of 1.4 to 3 (see Table 3). The indications of warfarin were: valve replacement (90 patients), deep vein thrombosis (5 patients), atrial fibrillation (5 patients), and stroke (4 patients). Clinical information (including age, sex, weight, and average daily maintenance dose) was obtained from every participant. At the time of genotyping, every patient had a constant maintenance dose for at least three weeks. Patients with liver, kidney, gastro-intestinal cancer, or abnormal bleeding problems before warfarin therapy were excluded.

TABLE 2
Patients demographics and genotypes

| Case | Dose | | Age | | Weight | VKORC1 | VKORC1 | #CYP2C9 |
|---|---|---|---|---|---|---|---|---|
| No. | (mg/d) | INR | (yr) | Sex | (kg) | −1639 | 1173 | variants |

Warfarin sensitive group (dose ≦ 1.5 mg/day, n = 11)

| | | | | | | | | |
|---|---|---|---|---|---|---|---|---|
| 1 | 0.71 | 3.23 | 65 | F | 42 | AA | TT | CYP2C9*3 |
| 2 | 1 | 2.69 | 74 | M | 65 | AA | TT | normal |
| 3 | 1 | 3.23 | 70 | M | 66 | AA | TT | CYP2C9*3 |
| 4 | 1.25 | 1.5 | 84 | F | 50 | AA | TT | normal |
| 5 | 1.25 | 1.96 | 68 | M | 75 | AA | TT | CYP2C9*3 |
| | | | | | | | | 895A > G(T299A) |
| 6 | 1.25 | 2.5 | 71 | F | 70 | AA | CT | 1145C > T(P382L) |
| 7 | 1.25 | 2 | 71 | F | 80 | AA | TT | normal |
| 8 | 1.25 | 2.9 | 72 | F | 67 | AA | TT | normal |
| 9 | 1.25 | 1.59 | 67 | M | 58 | AA | TT | normal |
| 10 | 1.43 | 2.05 | 58 | F | 42 | AA | TT | nonnal |
| 11 | 1.5 | 2.24 | 61 | M | 65 | AA | TT | normal |

Warfarin resistant group (dose ≧ 6 mg /day, n = 5)

| | | | | | | | | |
|---|---|---|---|---|---|---|---|---|
| 12 | 6.07 | 2.82 | 48 | F | 52 | AG | CT | normal |
| 13 | 8.57 | 2.09 | 63 | F | 58 | GG | CC | normal |
| 14 | 8.75 | 2.32 | 26 | M | 88 | GG | CC | normal |
| 15 | 10 | 1.3 | 46 | M | 64 | AG | CT | normal |
| 16 | 10 | 2.33 | 58 | F | 61 | GG | CC | normal |

MALDI-TOF mass spectrometry (SEQUENOM MassARRAY system, (Sequenom, San Diego, Calif., USA) was used to examine the polymorphism at position −1639 of the VKORC1 gene in the 104 Chinese patients receiving warfarin, 95 normal Chinese controls and 92 normal Caucasian controls. Briefly, primers and probes were designed using the SpectroDESIGNER software (Sequenom). Muliplex PCR was performed, and unincorporated dNTPs were dephosphorylated using shrimp alkaline phosphatase (Hoffman-LaRoche, Basel, Switzerland), followed by primer extension. The purified primer extension reactions were spotted onto a 384-element silicon chip (SpectroCHIP, Sequenom), analyzed in the Bruker Briflex III MALDI-TOF SpectroREADER mass spectrometer (Sequenom), and the resulting spectrum was processed with SpectroTYPER (Sequenom).

The association between the polymorphism at position −1639 of the VKORC1 gene and warfarin dosage did not change with respect to age, sex, and INR (see Table 3). Only two patients were found to carry homozygous GG at position −1639 and were grouped together with patients carrying AG at this position for statistic analysis, which was carried out without considering factors, such as diet or other medications. As shown in Table 3, patient carrying homozygous AA at position −1639 require group lower warfarin (2.61 mg/day) than patients carrying AG or GG at this position (3.81 mg/day). The differences were significant by either T test (p<0.0001) or Wilcoxon Mann Whitney test (p=0.0002) between the AA and AG/GG groups.

TABLE 3
Mean doses and other clinical characteristics of randomly selected
patients on warfarin stratified according to the genotypes.

| Genotype | Warfarin dose. | | | Sex |
|---|---|---|---|---|
| VKORC1˜1639 | (mg/day) | INR | Age (Years) | (M/F) |
| AA (n = 83) | 2.61 ± 1.10^{a} | 2.03 ± 0.45 | 57.5 ± 14.8 | 43/40 |
| AG + GG (n = 21) | 3.81 ± 1.24 | 2.08 ± 0.47 | 60.4 ± 13.1 | 13/8 |

^{a}P value of comparison between AA and AG + GG groups. P-value < 0.0001 using T test.
P-value = 0.0002 using Wilcoxon Mann Whitney test. Data represent mean ± SD.

### EXAMPLE 3

### Frequencies of VKORC1 −1639 G>A Polymorphism and CYP2C9 Variants in Chinese and Caucasians

It is well known that the Chinese population requires a much lower warfarin maintenance dose than the Caucasian population. To test whether differences in the VKORC1 −1639 genotype frequencies could account for the inter-ethnic differences in warfarin dosages, 95 normal Han Chinese subjects and 92 normal Caucasian subjects were genotype following the methods described above. In the Caucasian population, homozygous AA at position −1639 of the VOKRC1 gene had the lowest frequency, while the AG and GG genotypes made up the majority of the population (AA: 14.2%; AG: 46.7%; and GG: 39.1%).

In contrast, homozygous AA at position −1639 of the VKORC1 gene made up the majority of the Chinese population (i.e., 82.1%), while the rest of the population carrying heterozygous AG (i.e., 17.9%). Homozygous GG was not found in the selected Chinese patients. This AA/AG/GG was similar in the 104 warfarin patients in which 79.8% carry homozygous AA, 18.3% carry heterozygous AG, and the remaining 1.8% carry homozygous GG.

The genotype frequencies of each SNP were counted. See Table 4. The Chi-square test was used to compared genotype frequencies of each SNP for the three sample groups. T-test and Wilcoxon-Mann-Whitney test were performed for multiple comparisons of mean dose levels among the different genotype groups. Inter-marker linkage disequilibrium was assessed by two measures, D′ and r², calculated using Graphical Overview of Linkage Disequilibrium (GOLD, http://www.sph.umich.edu/csg/abecasis/GOLD/).

This frequency difference between the Caucasian group and the Chinese groups were significant (p<0.0001). It is also consistent with the clinical observation that the Chinese population requires a lower warfarin dose than the Caucasian population, given that homozygous AA is associated with warfarin sensitivity.

TABLE 4
Genotype frequencies of VKORC1 polymorphism (−1639 G < A)
and CYP2C9 variants in Chinese and Caucasians.

| | | | Random Selected |
|---|---|---|---|
| | Caucasian | | Chinese Warfarin |
| Genotype | (n = 92) | Chinese (n = 95) | Patients (n = 104) |
| VKGRC1 −1639 | | | |
| AA | 13 (14.2%) | 78 (82.1%) | 83 (79.8%) |
| AG | 43 (46.7%) | 17 (17.9%) | 19 (18.3%) |
| GG | 36 (39.1%) | 0 (0%) | 2 (1.9%) |
| CYP2C9 variants^{a} | | | |
| 2C9*1*2 | 20.4% | 0 (0%) | 0 (0%)^{b} |
| 2C9*2*2 | 0.9% | 0 (0% | 0 (9%)^{b} |
| 2C9*1*3 | 11.6% | 7 (7.3%) | 4 (5.4%)^{b} |
| 2C9*3*3 | 0.4% | 0 (0%) | 0 (0%)^{b} |

P value < 0.0001 compared between Caucasian anti Chinese population.
P value < 0.0001 compared between Caucasian and Chinese random selected warfarin patients.
P value = 0.817 compared between Chinese and random selected warfarin patients.
^{a}CYP2C9*1 is wild type of CYP2C9. CYP2C9*2 and CYP2C9*3 are variants with cysteine substitutes for arginine at residue 144 and leucine substitutes for isoleucine at residue 359, respectively. Frequencies in Caucasian were obtained from published data (17).
^{b}CYP2C9*3 frequency was derived from genotyping 74 warfarin patients.

In addition to −1639 G>A, three intronic polymorphisms (rs9934438, intron 1 1173 C>T; rs8050894, intron 2 g.509+124C; and rs2359612, intron 2 g.509+837C) and one 3′ UTR polymorphism (rs7294, 3730 G>A) were found in the Chinese population. All of them were in linkage disequilibrium with the −1639 promoter polymorphism (Inter-marker D′ and r2 values=1.0).

CYP2C9 variants were also examined in the Chinese groups. The frequency of CYP2C9*1/*3 was 7.3% in the Chinese control group and 5.4% in the randomly selected Chinese patients receiving warfarin. CYP2C9*2 variant was not detected in both the Chinese patients and controls. Compared to the published data on the CYP2C9 variant frequencies in Caucasians, the Caucasian population has a much higher frequency of CYP2C9 variants (*2 and *3) than Chinese (˜30% versus 7%), yet Caucasians are more resistant to warfarin. Other missense mutations detected in the warfarin sensitive patients, 895 A>G (T299A) and 1145 C>T (P3821L) (Table 2) were not found in any of the randomly selected patients and controls, suggesting that these were rare mutations.

### EXAMPLE 4

### VKORC1 Promoter Activity

To analyze the VKORC1 promoter activity, the promoters from patients with −1639 AA and −1639 GG genotypes were PCR amplified using the forward primer: 5′-ccgctcgagtagatgtgagaaacagcatctgg (SEQ ID NO:______; containing an XhoI restriction site) and the reverse primer: 5′-cccaagcttaaaccagccacggagcag (SEQ ID NO:_______; containing a HindIII restriction site). The PCR products were then cloned into the pGEM-T Easy vector (Promega, Madison, Wis., USA). The fragments containing the VKORC1 promoters were released from the pGEM-T Easy vector by XhoI and HindIII digestion and sub-cloned into the pGL3-basic vector (XhoI and HindIII) (Promega). The pGL-3 vector contains the cDNA encoded for firefly luciferase, which when fused with a potential promoter fragment, can be used to analyze the promoter activity of the inserted fragment upon transfection into mammalian cells. The vector containing the promoter fragment carrying −1639 G/G was designated pGL3-G and the vector containing the promoter carrying −1639 A/A was designated pGL3-A. VKORC1 promoter sequences in both vectors were confirmed by direct sequencing analysis.

HepG2 cell (a human hepatoma cell line) was chosen for the promoter assay since VKORC1 is expressed at the highest level in the liver. HepG2 cells were grown in Dulbecco's modified Eagle medium (DMEM) and 10% fetal calf serum supplemented with 100 units/mL Penicillin, 100 μg/mL Streptomycin and 2 mM L-Glutamine. Twenty-four hours prior to transfection, 1.5×10⁵ cells were seeded in each well in a 12-well plate. Cells in each well was co-transfected with 1.5 μg of either pGL3-G or pGL3-A, and 50 ng of the pRL-TK vector (Promega) using lipofectamine 2000 (Invitrogen Corporation, Carlsbad, Calif., USA). The pRL-TK vector encodes Renilla luciferase, whose expression is driven by HSV-TK promoter. This vector was used as an internal control of normalize firefly luciferase expression. Forty-eight hours after transfection, the cells were lysed in passive lysis buffer (Promega) and luciferase substrates (dual luciferase reporter system, Promega) were added to the cell lysate. The Firefly and Renilla luciferase activities were measured with a luminometer (SIRIUS, Pforzheim, Germany).

A total of 9 experiments were performed, and all demonstrated consistent results (shown in FIG. 2). The cells transfected with −1639 G VKORC1 promoter showed higher luciferase activity (approximately 44% higher) than cells transfected with the −1639 A promoter. These data demonstrate that the nucleotide at position −1639 of the VKORC1 gene is important for its promoter activity, and higher promoter activity (e.g., −1639G) is associated with higher warfarin dose requirements.

### EXAMPLE 5

### Warfarin Dosing Based on the Combination of the VKORC1 −1639 SNP and the CYP2C9 Genotype

160 patients were participated in this study and data obtained from 108 patients were used in the final analysis. 52 patients were excluded from the study for the following reasons: (1) 15 patients did not return for follow up after signing the consent or have their consents withdrawn during the study, (2) three patients were diagnosed for lung cancer during the course of this study, (3) 11 patients showed poor compliance which included failure to return for regular follow up visit and failure to take the prescribed warfarin dose daily as determined by patient's own admission and PIVKA-II measurements, (4) for 23 patients, initial warfarin doses were prescribed before their genotyping results were available or doses were prescribed without considering their genotypes. Demographic characteristics of the patients are shown in Table 6. The recruited patients consisted of the elderly population with mean age of 64.4±13.6 and male patients made up more than half of the patients recruited (58%). 69% of the patients in this study were on warfarin therapy due to atrial fibrillation followed by deep vein thrombosis (16%), stroke (10%) and heart valve replacement (5%). Body surface area estimated from height and weight was 1.68±0.18 m². Of all the patients recruited, 19% had diabetes, 54% had hypertension and 18% had poor ventricular function. Only a small proportion reported to consume alcohol on daily basis (7%).

The initial warfarin dosage for a patient was predicted based on the nucleotide at position −1639 of the VKORC1 gene and the genotype of CYP2C9 gene of that patient.

10 ml of blood was drawn from each patient using sodium citrate tubes. Blood samples were centrifuged at 3,000 g for 10 minutes to separate the plasma. Plasma and the packed cells were transferred to National Genotyping Center, Institute of Biomedical Sciences, Academia Sinica for storage and genomic DNA extraction. Genomic DNA was extracted using PUREGENE™ DNA purification system (Gentra Systems, Minnesota, USA). VKORC1 and CYP2C9 genotypes were determined using PCR-RFLP. CYP2C9*3 RFLP primers used have been reported previously. See Sullivan-Klose et al., 1996. The RFLP primers for the VKORC1 −1639 A>G polymorphism were: 5′-GCCAGCAGGAGAGGGAAATA-3′SEQ ID NO:______, forward primer and 5′-AGTTTGGACTACAGGTGC CT-3′SEQ ID NO:______, reverse primer. A 290 bp fragment was generated using these primers. The VKORC1 −1639 G allele created on MspI restriction site and resulted in 123 bp and 167 bp fragments upon MspI digestion. The PCR reaction was carried out in a final volume of 50 μl, containing 0.4 μM of each primer, 0.2 mM dNTPs, 1.5 mM MgCl₂, 50 mM KCl, 10 mM Tris HCl (pH9.0) and 1% Triton X-100 buffer, 2.5 unit Taq DNA polymerase (MDBio, Taiwan). The results from the PCR-RFLP were reported to the physicians within 48 hours of blood collection and were later verified by direct sequencing within one week.

The entire exons, exon-intron junctions and up to 1 kb promoter regions of VKORC1 and CYP2C9 genes were also sequenced in those patient with maintenance dose did not matched the predicted dose. See Yuan et al., 2005.

As expected, wild type CYP2C9 and VKORC1 homozygous −1639 AA made up the majority (76.9%) of the recruited patients. Patients carrying wild type CYP2C9 and heterozygous VKORC1 −1639 AG constitute 15.7% of the total patients, patients carrying homozygous −1639 AA and CYP2C9*3 constitute 3.7%, and patients carrying homozygous −1639 GG and wild type CYP2C9 2.8%. Homozygous CYP2C9*3 was not found in any of the recruited patients due to its low frequency in Chinese.

Each patients was then administered with Couimadin (a warfarin) at an initial dosage as predicted based on his or her genotype, see Table 5.

TABLE 5
Suggested initial warfarin dose based on CYP2C9 and VKORC1 genotypes

| VKORC1 | | Genetic Based | Expected | Actual Frequency |
|---|---|---|---|---|
| −1639 G > A | CYP2C9 | Dose (mg/d) | frequency (%) | (n = 108) |
| GG | *1/*1 | 5 | 1.8 | 3 (2.8%) |
| GG | (*1/*3) or *(1/*2) | 3.75 | 0.1 | 0 |
| GG | (*2 or *3)/(*2 or *3) | 3.75 | <0.01 | 0 |
| AG | *1/*1 | 3.75 | 17.3 | 17 (15.7%) |
| AG | (*1/*3) or *(1/*2) | 2.5 | 1 | 1 (0.9%) |
| AG | (*2 or *3/(*2 or *3) | 2.5 | <0.01 | 0 |
| AA | *1/*1 | 2.5 | 75.5 | 83 (76.9%) |
| AA | *1/*3 or *(1/*2) | 1.25 | 4.3 | 4 (3.7%) |
| AA | (*2 or *3)/(*2 or *3) | 1.25 | <0.01 | 0 |

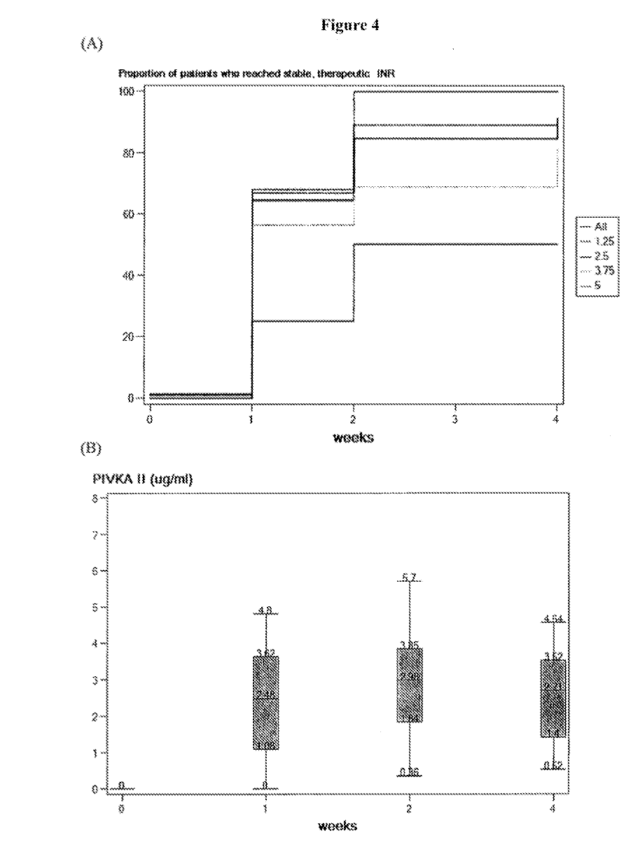

INR for these patients was monitored at each follow-up (once per week) to ensure that overdose of warfarin would not occur. Before the treatment began, the average INR for all the participants was 1.06±0.11 ranged from 0.89 to 1.46. As shown in FIG. 4A, 85% of all the patients reached the therapeutic INR of 1.7 and 3 within the first two week of treatment initiation. However, only 50 % of the 12.5 mg/d dose group reached the therapeutic INR at week 2, with the other half required more than 5 weeks reaching the therapeutic INR. 11 individuals (all CYP2C9 wild type) had INR greater than 4 for one week during the course of the study, however, four of these events were due to concomitant medication (Amiodarone and rosuvastation) or the use of Chinese herbal medicine. Once these factors were removed, these patients' INR quickly returned to normal. Averse events (bleeding episodes or venous thrombosis/pulmonary embolism) were absent during the study.

Patients' vitamin K status was determined by measuring PIVKA-II before and during warfarin treatment to assess how the treatment affected physiological vitamin K concentration. PIVKA-II is an abnormal decarboxylated prothrombin, which is present in vitamin K deficiency or in patients using warfarin. Plasma was separated from whole blood within 30 minutes after blood draw and stored in 31 80° C. freezers until measured. PIVKA-II concentration was measured using a murine monoclonal antibody available in an enzyme immunoassay kit according to manufacturer's instructions. (Asserachrom PIVKA-II; Diagnostica-Stago, Asnieres Sur Seine, France). The normal value for PIVKA-II in adults was <2 ng/mL with this method.

PIVKA-II was virtually undetectable before treatment (<2 ng/mL), however, 4 cases had detectable PIVKA-II (>0.15 ug/ml) indicating they could have vitamin K deficiency. The average PIVKA-II during the course of the treatment is shown in FIG. 4B. As expected, PIVKA-II increased after treatment indicating a decreased in vitamin K caused by warfarin inhibition on PIVKA-II. 10 patients (˜10%) however, had PIVKA-II levels decreased during the course of study indicting non-compliance. The average PIVKA-II concentration at the end of the study was 2.5 ug/mL.

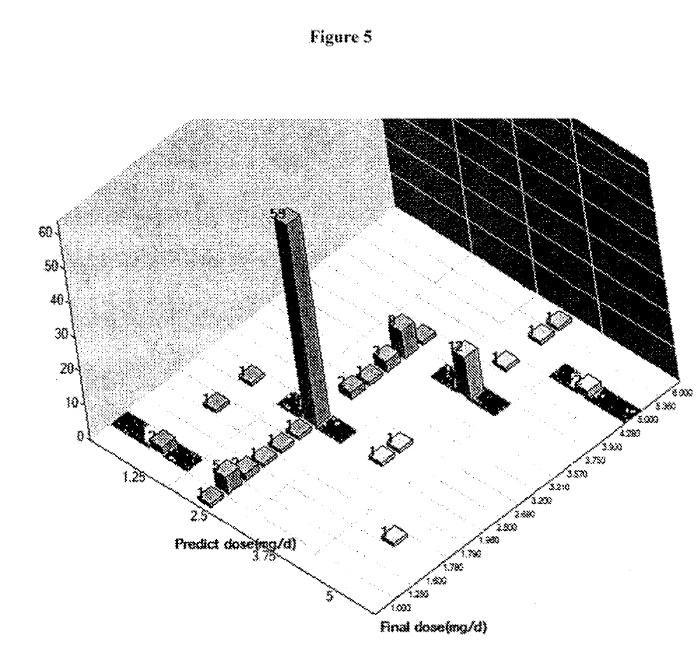

At 12 weeks of follow-up, the average maintenance dose of the recruited patients was 2.76±0.88 mg/d with the dose ranging from 1 mg/d to 6 mg/d. FIG. 5 shows the correlation between the predicted and maintenance dose. The shaded area denotes where the maintenance dose match the predicted dose. The doses prescribed based on the genotypes were non-continuous (1.25, 2.5, 3.75 and 5 mg/d). In addition, physicians usually alternate doses when adjusting warfarin. Therefore, final doses within 0.5 mg/d of the predicted dose were considered as matching the predicted doses. Approximately 69% of the recruited patient's maintenance dose matched their predicted doses. This result shows that the genotype dosing strategy can predict warfarin dosage with high accuracy in patients requiring both low (1.25 mg/d) and high (3.75 and 5 mg/d) doses.

### EXAMPLE 6

### Effects of Non-Genetic Factors on Warfarin Dosage

To access the influence of non-genetic factors in warfarin dosing, univariate analyses was performed on the patients described above with respect to their predicted dose (determined from genotypes), age, gender, diabetes, hypertension, poor ventricular function, warfarin indication, body surface area, alcohol consumption and concomitant medications. These patients' demographics data are shown in Table 6. Body surface area estimated from height and weight was 1.68±0.18 m2. Of all the patients recruited, 19% had diabetes, 54% had hypertension and 18% had poor ventricular function. Only a small proportion reported to consume alcohol on daily basis (7%).

The results showed significant association of the maintenance dose with genotypes, age and body surface area. Other non-genetic factors, such as poor ventricular function, and gender, did not contribute to warfarin dosage significantly as described in previous studies. Since patients from one single hospital made up the majority of the recruited population (70%), the factors were re-analyzed using patients only recruited from this hospital (N=75) and results are shown in Table 7B. Again, the results demonstrated the significant association of genotypes, body surface area, and age with warfarin dosage. however, Medical conditions, e.g., hypertension, also affects warfarin dosage, albeit to a lesser extent. In order to increase the prediction accuracy, a dosing algorithm was generated using regression analysis based on factors shown to affect warfarin dose in this study. The multiple regression model included predicted dosage based on genotypes (genetic-based), age and body surface area: Dose=−0.432+0.769×genetic-based dosage−0.015×Age+1.125×body surface area. These factors in this model accounted for 48.2% of the variation as measured by R2. Using the samples from the same hospital, the model below was generated: Dose=−0.443+0.798×genetic-based dosage−0.018×age+1.4×body surface area−0.269×HT with R2 of 0.62.

TABLE 6
Patient demographics

| | |
|---|---|
| Variable | n = 108 |

| | |
|---|---|
| Age, y, mean ± SD (range) | 64.4 ± 13.6 (19.0-88.0) |
| Sex, n (%) | |
| Male | 63 (53) |
| Female | 45 (42) |
| BSA, mean ± SD (range) | 1.68 ± 0.13 (1.22-2.37) |
| Concomitant medication, n (%) | |
| Yes | 21 (99) |
| No | 88 (81) |
| Diabetes mellitus, n (%) | |
| Yes | 21 (19) |
| No | 87 (81) |
| Hypertension, n (%) | |
| Yes | 58 (54) |
| No | 50 (46) |
| Poor ventricular function, n (%) | |
| Yes | 19 (18) |
| No | 89 (82) |
| Indication, n (%) | |
| Artial fibrillation | 75 (69) |
| Stroke | 11 (10) |
| Deep vein thrombosis | 17 (16) |
| Cardiac valve replacement | 5 (5) |
| Alcohol, n (%) | |
| Yes | 8 (7) |
| No | 100 (93) |

BSA=Body Surface Area (m²) Concomitant medication: aminodarone (8), simvastatin (1), allopurinol (1), acetaminophen (2), rosuvastatin (1), phenyton (1), NSAID (2), gemfibrozil (1), fluvastatin (1), fenofibrate (1), atorvastatin (1), carbamazepine (1).

TABLE 7
Factors affecting warfarin dose requirements in regression models

| Variable | R2 × 100% | P value |
|---|---|---|
| (A) | | |
| Predicted dose | 33.4 | <0.001 |
| (genotype) | | |
| BSA | 9.7 | <0.001 |
| Age | 5.1 | 0.002 |
| Indication | 0.8 | 0.20 |
| Alcohol | 0.7 | 0.25 |
| Diabetes mellitus | 0.6 | 0.27 |
| Concomitant | 0.12 | 0.63 |
| medication | | |
| Hypertension | 0.06 | 0.73 |
| Poor ventricular | <0.001 | 0.94 |
| function | | |
| Gender | <0.001 | 0.98 |
| (B) | | |
| Predicted dose | 36.3 | <0.001 |
| (genotype) | | |
| Age | 17.4 | <0.001 |
| BSA | 6.5 | 0.001 |
| Hypertension | 2.1 | 0.05 |

(A): Univariate regression analysis of the factors influencing warfarin dose.
(B): Univariate regression analysis for patients from a single hospital where 70% of patients were recruited (CGMH) (N = 75); only the significant variables were listed.

### EXAMPLE 7

### Determining SNP at position −1639 of the VKORC1 Gene and Genotype of the CYP2C9 Gene

Real Time PCR:

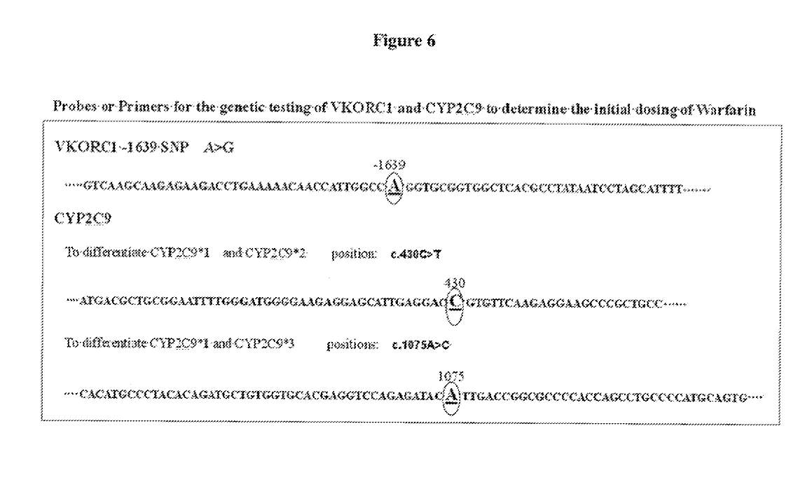

Genomic DNAs were extracted from a patient's blood or saliva. PCR primers designed to amplify regions that include the nucleotides of interests, i.e., at position −1639 of the VKORC1 gene, at position c.430 of the CYP2C9 gene, or at the position of c.1075 of the CYP2C9 gene (see FIG. 6), were synthesized. The PCR amplification was carried out as follows: (i) activating polymerase at 95° C. for 4.5 minutes, (ii) denaturing DNA template at 92° C. for 15 seconds and annealing/elongating DNA chains at 60° C. for 90 second, and (iii) conducting 38 cycles of denaturing/annealing/elongating.

A pair of TaqMan probes for detecting the SNP at position −1639 were synthesized, The probe for detecting −1639 G was labeled with FAM, and the probe for detecting −1639 A was labeled with VIC.

TaqMan probes for detecting CYP2C9 variants were also synthesized. Two probes each targeting the c.430 C allele or the c.430 T allele of the CYP2C9 gene were designed to determine whether a patient carries CYP2C9 *1 or CYP2C9 *2. One of the two probes was labeled with fluorescent dyes VIC and the other with FAM. Two probes each targeting the c.1075 A allele or the c.1075 C allele were designed to determine whether a patient carries CYP2C9 * 1 or CYP2C9 *3. One of them was labeled with VIC and the other with FAM. In addition to the fluorescent dyes, all of the probes were also labeled with a quencher moiety. When a probe perfectly matches an allele, the dye would release fluorescent signals. When the match is not perfect, the fluorescent signals would be quenched by the quencher moiety.

The presence or absence of an allele (containing a nucleotide of interest) was determined based on the threshold cycle (Ct) value. A Ct value of 20-30 indicates the presence of a specific allele and a Ct value greater than 35 indicates the absence of the specific allele. If an allele is homozygous, a substantial increase of the signal released by either VIC or FAM is expected. If an allele is heterozygous, substantial increases of the signals released by both the dyes are expected.

Genomic DNA samples were prepared from 131 Han Chinese patients, and 100 Caucasian patients. The SNP at position −1639 of the VKORC1 gene and the genotype of the CYP2C9 gene were determined by real time PCR, and the data were verified by direct sequencing. The results indicate that the specificity and sensitivities of this method were >99%.

PCR-RFLP:

RFLP stands for restriction fragment length polymorphism, which has the advantages of requiring relatively low cost and time (only takes about 12 hours). In addition, it does not need expensive equipments. Exemplary procedures of this method are described below:
(1) Genomic DNA extraction: Genomic DNA is extracted from a patient (e.g., whole blood, saliva, and serum) using methods known in the art.
(2) PCR: PCR products containing an allele of interest can be obtained using properly designed primers and the genomic DNA as a template. A pair of exemplary VKORC1 primers are shown in Table 8, and pairs of exemplary CYP2C9 primers are shown in Table 9 below.
(3) Restriction Enzyme Digestion: the amplified PCR products are subjected to restriction enzyme digestion. For example, PCR products containing position −1639 of the VKORC1 gene are digested with the restrictions enzyme MspI; PCR products containing position c.430 and c.1075 of CYP2C9 are digested with NsiI and KpnI, respectively.

(4) Genotype Determination: The SNP of VKORC1 at −1639 (A/G) and the subtype of CYP2C9 are determined according to the patterns of fragmentations.

TABLE 8

Examples of PCR-RFLP primers for VKORC1:

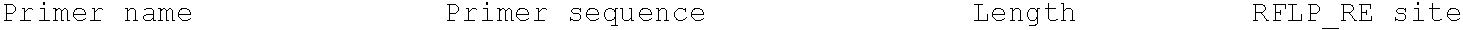

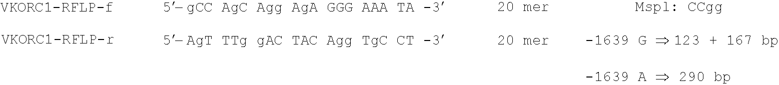

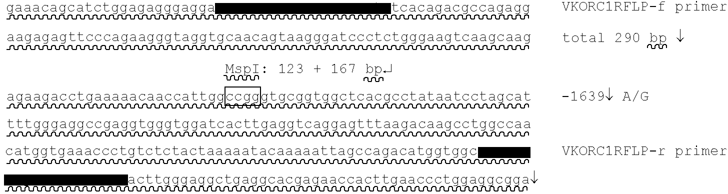

TABLE 9

Mismatched PCR-RFLP primer for CYP2C9:

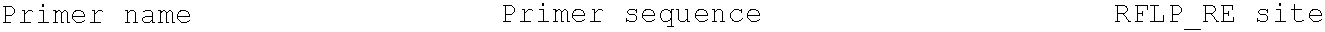

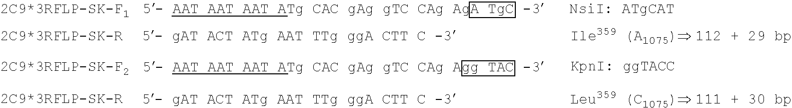

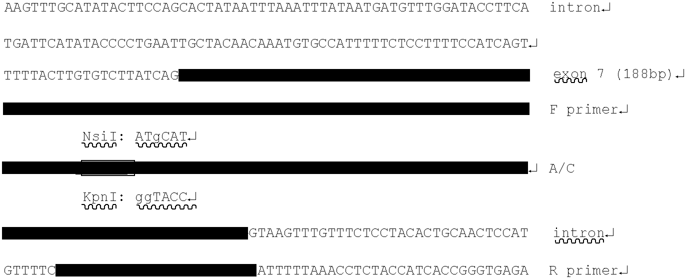

DHPLC:

DHPLC stands for Denaturing High-Performance Liquid Chromatography (DHPLC), which can identify mutations by detecting sequence variation in re-annealed DNA strands (heteroduplexes). This method efficiently directly detects single nucleotide and insertion/deletion variations in crude PCR products without DNA sequencing. The type of polymerase used will affect the analysis of the samples. An exemplary procedure includes the following steps:
(1) Genomic DNA extraction: Genomic DNA is extracted from a patient (e.g., whole blood, saliva, and serum) using methods known in the art.
(2) PCR: PCR products containing an allele of interest can be obtained using properly designed primers and the genomic DNA as a template. A pair of exemplary CYP2C9 primers are shown in Table 10, and pairs of exemplary VKORC1 primers are shown in Table 11 below.

(3) Genotype determination: The SNP for VKORC1 at −1639 (A/G) and the subtype of CYP2C9 are determined through the pattern of PCR products by Denaturing High-Performance Liquid Chromatography analysis.

TABLE 10

DHPLC primers for CYP2C9 subtype determination

| | | | | SNP site/ |
|---|---|---|---|---|
| | | Primer | Region of | PCR product |
| Primer name | Primer sequence | length | Analysis | size |
| CYP2C9exon7_dhplc-MD-F1 | Forward primer: | 23 | CYP2C9 exon7 | 178/276 bp |
| | 5′- GAATTGCTACAACAAATGTGCCA -3′ | | | |
| CYP2C9exon7_dhplc-MD-R1 | Reversed primer | 24 | | |
| | 5′- GCAGTGTAGGAGAAACAAACTTAC - | | | |
| | 3′ | | | |

TABLE 11

DHPLC primers for VKORC1 SNP

| | | | | SNP site/ |
|---|---|---|---|---|
| Primer | | Primer | | PCR product |
| name | Primer sequence | length | Region of Analysis | size |
| VKORC1 | 5′- CAAgTTCCAgggATTCATgC -3′ | 20 | VKORC1 promoter region | 229/465 bp |
| | 5′- gTgCCATCTCggCTCACT -3′ | 18 | | |
| VKORC1 | 5′- GCCAGCAGGAGAGGGAAATATCA - | 23 | VKORC1 promoter region | 124/272 bp |
| | 3′ | | | |
| | 5′- CTGCCACCATGTCTGGCTAATTT -3′ | 23 | | |
| VKORC1 | 5′- TATTCTGTCTACCACACTCTCTA -3′ | 23 | VKORC1 promoter region | 188/361 bp |
| | 5′- CCCAAGTAGTTTGGACTACAGGT -3′ | 23 | | |

CSSO-ELISA:

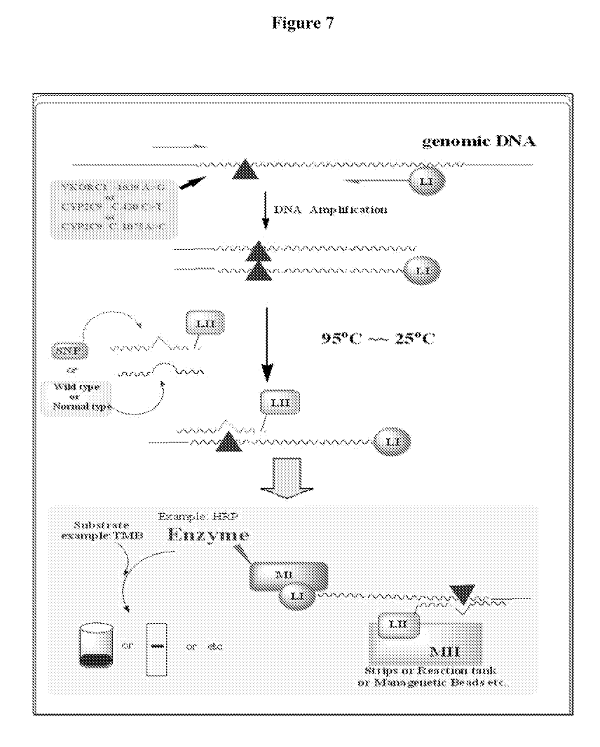

An exemplary procedure is described below (also shown in FIG. 7.)
(1) DNA amplification: Genomic DNAs were purified from patients following methods known in the art. The genomic DNAs were first amplified using methods used for whole genomic amplification (WGA). PCR products containing the position −1639 of VKORC1 and the positions of c.430 and c.1075 of the CYP2C9 genes were amplified either from the genomic DNAs directly, or from the amplified genomic DNAs, using properly designed primers. Among a pair of the primers for amplifying a PCR product described above, either the forward primer or the reverse primer was labeled with a Ligand I (LI), which is recognizable by Molecular I labeled with an Enzyme, e.g., HRP.
2) Competitive Hybridization: Two competitive sequencing specific oligonucleotides were designed, each targeting a particular polymorphism of either the −1639 SNP in the VKORC1 gene or the CYP2C9 gene. One of the two oligonucleotides was labelled with Ligand II (LII), which is recognizable by Molecule II. These oligonucleotides were hybridized with the PCR products under stringent hybridization conditions.
(3) Immobilization: The oligonucleotides were immobilized on a reaction tank, a strip, or a number of magnetic beads through the interaction between Ligand II and Molecule II. The PCR products, when hybridized with the immobilized oligonucleotides, were thus captured.
(4) Enzyme-linked Colorimetric Development: The presence of the PCR products were determined by enzyme-linked assays.

### OTHER EMBODIMENTS

All of the features dislcosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of predicting dosage of a warfarin for a patient, the method comprising:
determining the nucleotide at position −1639 of the VKORC1 gene of the patient,
examining the sequence of the CYP2C9 gene of the patient, and
predicting the warfarin dosage for the patient based on the nucleotide at position −1639 of the VKORC1 gene and the CYP2C9 gene sequence.

2. The method of claim 1, wherein the predicted warfarin dosage ranges from 4.5 to 6.5 mg per day if the patient has homozygous GG at position −1639 of the VKORC1 gene and has CYP2C9*1/*1.

3. The method of claim 1, wherein the predicted warfarin dosage ranges from 3.25 to 4.25 mg per day if the patient has homozygous GG at position −1639 of the VKORC1 gene and has CYP2C9*1/*3 or CYP2C9*1/*2.

4. The method of claim 1, wherein the predicted warfarin dosage ranges from 3.5 to 4.0 mg per day if the patient has homozygous GG at position −1639 of the VKORC1 gene and has CYP2C9*2/*2, or CYP2C9*3/*2, or CYP2C9*3/*3.

5. The method of claim 1, wherein the predicted warfarin dosage ranges from 3.25 to 4.05 mg per day if the patient has homozygous AG at position −1639 of the VKORC1 gene and has CYP2C9*1/*1.

6. The method of claim 1, wherein the predicted warfarin dosage ranges from 2.5 to 3.0 mg per day if the patient has homozygous AG at position −1639 of the VKORC1 gene and has CYP2C9*1/*3 or CYP2C9*1/*2.

7. The method of claim 1, wherein the predicted warfarin dosage ranges from 2.0 to 3.0 mg per day if the patient has homozygous AG at position −1639 of the VKORC1 gene and has CYP2C9*2/*2, CYP2C9*3/*2, or CYP2C9*3/*3; or if the patient has homozygous AA at position −1639 of the VKORC1 gene and has CYP2C9*1/*1.

8. The method of claim 1, wherein the predicted warfarin dosage ranges from 1.0-1.5 mg per day if the patient has homozygous AA at position −1639 of the VKORC1 gene and has CYP2C9*1/*3 or CYP2C9*1/*2.

9. The method of claim 1, wherein the predicted warfarin dosage ranges from 0.75 to 1.5 mg per day if the patient has homozygous AA at position −1639 of the VKORC1 gene and has CYP2C9*2/*2, CYP2C9*3/*2, or CYP2C9*3/*3.

10. The method of claim 1, further comprising adjusting the predicted warfarin dosage based on the patient's age, body surface area, medical condition, or use or non-use of a drug that interferes with CYP2C9 activity or affects VKORC1 expression, or a combination thereof.

11. The method of claim 10, wherein the warfarin dosage is determined as follows: A+(B×genetic-based dosage)+)C×age)+(D×body surface area), wherein A is in the range of −2 to 0, B in the range of 0.5 to 1.0, C in the range of −0.1 to 0.015, D in the range of 0 to 5.

12. The method of claim 11, wherein B is in the range of 0.7 to 0.8, C in the range of −0.05 to 0.01, D in the range of 0.5 to 1.5.

13. The method of claim 12, wherein the warfarin dosage is determined as follows: −0.432+(0.769×genetic-based dosage)−(0.015×age)+(1.125×body surface area).

14. The method of claim 11, wherein A is adjusted based on the patient's medical condition or use or non-use of a drug that interferes with CYP2C9 activity or affects VKORC1 expression

15. The method of claim 14, wherein the medical condition is hypertension or diabetes.

16. The method of claim 14, wherein the drug is a drug for treating a cardiovascular disease or hypercholesterolemia,

17. The method of claim 16, wherein the drug is aminodarone or rosuvastatin.

18. A method for determining final dosage of a warfarin for a patient, the method comprising:
predicting a warfarin dosage based on the nucleotide at position −1639 of the VKORC1 gene and the genotype of the CYC2 C9 gene,
administering to the patient the warfarin at the predicted dosage,
monitoring the patient's therapeutic international normalized ratio (INR), and
adjusting the warfarin dosage until the INR falls in the range of 1.7 to 3;
wherein the dosage at which the patient's INR maintains in the range of 1.7 to 3 for at least two consecutive measurements is determined to be the final dosage for the patient.

19. A kit for predicting a warfarin dosage for a patient, the kit comprising:
a first probe for detecting the nucleotide at position −1639 of the VKORC1 gene, and
a second probe for detecting position c.1075 of the CYP2C9 gene.

20. The kit of claim 19 further comprising a third probe for detecting position c.430 of the CYP2C9 gene.

21. The kit of claim 19, wherein the first and second probes are oligonucleotides.

22. The kit of claim 19, wherein the first or the second probe is a pair of PCR primers.
